(19) United States
(12) Patent Application Publication
(10) Pub. No.: US 2026/0234218 A1
(43) Pub. Date: Aug. 13, 2026

(54) APOLIPOPROTEIN E FOR THE TREATMENT OF MUSCLE INJURY

(71) Applicant: Virginia Commonwealth University, Richmond, VA (US)

(72) Inventors: Michael J. McClure, Richmond, VA (US); James T. Redden, Richmond, VA (US); Zvi Schwartz, Richmond, VA (US)

(21) Appl. No.: 19/326,259

(22) Filed: Sep. 11, 2025

Related U.S. Application Data
(60) Provisional application No. 63/693,917, filed on Sep. 12, 2024.

(51) Int. Cl. C07K 14/775; A61K 38/00

(52) U.S. Cl. CPC C07K 14/775; A61K 38/00

(57) ABSTRACT

Methods for treating a muscle injury, such as denervated muscle tissue, in a subject in need thereof include administering to the subject a therapeutically effective amount of a composition comprising apolipoprotein E (apoE) or a function-conservative variant thereof. ApoE is useful for maintaining the integrity of the neuromuscular junction prior to reconnection of an innervating nerve. Compositions containing apoE encapsulated in decellularized extracellular matrix or hydrogel can provide for targeted release at the site of the muscle injury.

### CROSS-REFERENCE TO RELATED APPLICATIONS

This application claims the benefit of priority to U.S. Provisional Patent Application Ser. No. 63/693,917 filed Sep. 12, 2024, which is incorporated by reference herein in its entirety.

### SEQUENCE LISTING

This document incorporates by reference an electronic sequence listing XML file, which was electronically submitted along with this document. The XML file is named 02941786AA_seqlisting.xml, is 4536 bytes, and was created on Sep. 10, 2025.

### FIELD OF THE INVENTION

The invention is generally related to the use of apolipoprotein E (apoE) for treating muscle injuries. In particular, apoE is useful for maintaining the integrity of the neuromuscular junction prior to reconnection of an innervating nerve.

### BACKGROUND OF THE INVENTION

Muscle endplate formation is a step-wise process requiring coordinated signaling leading to acetylcholine receptor (AChR) clustering, pruning of clusters, and innervation of the motor neuron onto each muscle fiber. Impairments to muscle-nerve cross-talk occur for a variety of clinical reasons including traumatic injuries (Dahlin and Wiberg, 2017; Noble et al., 1998), myasthenia gravis (MG) (Bubuioc et al., 2021), congenital myasthenic syndromes (CMS) (Engel et al., 2015), and peripheral neuropathies (Hicks and Selvin, 2019) leading to reduced patient quality of life. Despite several decades of research, much remains unknown about the underlying mechanisms required to form and support the neuromuscular junction (NMJ).

AChR clustering is dependent on low-density lipoprotein receptor-related protein 4 (Lrp4) and muscle specific kinase (MuSK) signaling (Kim et al., 2008). Neural-derived agrin is a well-established pathway that activates Lrp4-MuSK to study AChR aggregation in vitro, and has been the primary focus in elucidating AChR clustering (Choi et al., 2013; Gemza et al., 2022; Kim et al., 2008; Song and Balice-Gordon, 2008; Xie et al., 2023; Zhang et al., 2008; Zong et al., 2012). In these cited experimental designs, exogenous agrin was used to induce AChR clustering (Bowen et al., 1996; Nitkin et al., 1987; Tourovskaia et al., 2006) but was insufficient to stabilize the immature endplates leading to AChR resorption without continued treatment. Despite this, the model has been used to study endplate dynamics and subsequent signaling pathways, and a potential limitation of the neural agrin model is that it assumes the peripheral nerve is in immediate proximity. However, AChR clusters form prior to motor nerve invasion and involves a series of complex pathway activations that remain unclear. Understanding how motor end plates form can help identify targets that could bolster muscle regeneration and innervation in cases of trauma and disease.

Immature and aneural AChR clusters form prior to innervation during embryogenesis in an event called pre-patterning. These pre-patterns of AChRs are diffuse along the myotubes at much lower concentrations than AChR aggregations observed in adults (Sanes and Lichtman, 1999) and occur spontaneously during early myogenesis (Sanes and Lichtman, 2001). However, not all pre-patterned sites are used by the invading nerve. This results in a redistribution of the endplates as the newly invading axon signals to the muscle (Anderson and Cohen, 1977), producing poly-neuronal innervation and creating an over-abundance of NMJs along each muscle fiber (Redfern, 1970). To generate proper motor recruitment from each fiber, the endplates are pruned throughout early post-natal maturation to eliminate local axon branching until a singular high density AChR endplate and mono-innervating axon form the NMJ (Brown et al., 1976; Keller-Peck et al., 2001). The assembly of the postsynaptic motor endplate relies on pathway enrichment activated by Wnt (Barik et al., 2014; Hubbard and Gnanasambandan, 2013), neuregulin (Schmidt et al., 2011), agrin14,19, and acetylcholine (Dahm and Landmesser, 1988; Lin et al., 2005), but despite decades of research, unraveling the factors involved in synaptogenesis remains challenging.

New compositions and methods for treating muscle injuries, e.g. by reducing endplate fragmentation, are needed.

### SUMMARY

An aspect of the disclosure provides a method of treating a muscle injury in a subject in need thereof, comprising administering to the subject a therapeutically effective amount of a composition comprising apolipoprotein E (apoE) or a function-conservative variant thereof. In some embodiments, the muscle injury comprises denervated muscle tissue. In some embodiments, the method further comprises reconnecting an innervating nerve in the denervated muscle tissue after administration of the composition. In some embodiments, the composition comprises apoE or a function-conservative variant thereof encapsulated in a decellularized extracellular matrix. In some embodiments, the decellularized extracellular matrix is a decellularized muscle matrix. In some embodiments, the composition comprises a hydrogel containing apoE or a function-conservative variant thereof. In some embodiments, the composition comprises a nucleic acid encoding apoE or a function-conservative variant thereof. In some embodiments, the composition is injected or implanted at a site of the muscle injury. In some embodiments, the apoE is an apolipoprotein E2 (apoE2), apolipoprotein E3 (apoE3), or apolipoprotein E4 (apoE4) isoform. In some embodiments, the function-conservative variant has a sequence at least 90% identical to SEQ ID NO: 1.

### BRIEF DESCRIPTION OF THE DRAWINGS

FIGS. 1A-J. Validation of RNA sequencing using RT-qPCR. RNA sequencing counts were validated using RT-qPCR for (a) Mb, (b) Sppl, and (c) Apoe demonstrating that Apoe is elevated in skeletal muscle cells co-cultured with motor axons. (d) Adam RNA-seq counts were evaluated to validate Reactome pathway analysis and showed elevated Adam 10, Adam 17, and Adam 19. (e) The Erbb2 receptor was not affected. Sequencing data was validated for (f) Lrp5, (g) Fzd7, (h) Rhoa [unaffected], (i) Rac1, and (j) Jnk [unaffected].

FIGS. 2A-J. To test the effects of ApoE in vitro, C2C12 myoblasts were dosed with either ApoE3 or ApoE4 isoforms for six days following 100% confluence. (a, b) DNA levels were not affected by ApoE treatment. (c) Chrng expression dose dependently increased in C2C12 cells treated with ApoE3 (d) Chrng was unaffected by ApoE4. All other synaptogenic markers were unchanged by the ApoE3 and ApoE4 treatment (e, f, j).

FIGS. 3A-H. Myogenic associated genes were unaffected by ApoE3/4 treatment suggesting a synaptogenic specific effect of ApoE3 on myotubes. (a, b) Myodl, (c, d) Myog, (e, f) Itga7, and (g, h) Myhl were all evaluated and showed no changes regardless of ApoE dose.

FIGS. 4A-C. (a, b) Morphometrics showed myotube counts and widths were unchanged between treatments. (c) Synaptic counts were highest in 10⁻⁸ and 10⁻⁷ ApoE3 treated cells compared to vehicle and anti-Lrp4 only treated cells.

FIGS. 5A-B. (a) Peak twitch and (a) peak tetanic force was measured in WT, Apoe3 KI animals, and KO animals.

FIG. 6. Amount of human recombinant ApoE was adsorbed onto the surface of a decellularized muscle matrix (DMM) scaffold.

FIG. 7. Rate of force decline in WT, ApoE3 knock-in (ApoE3 KI), and ApoE knock-out (ApoE KO) mice.

### DETAILED DESCRIPTION

Embodiments of the disclosure provide the use of apolipoprotein E (apoE) or a function-conservative variant thereof for the treatment of a muscle injury.

The human APOE gene encodes the glycoprotein apolipoprotein E which functions as a high-affinity ligand facilitating the clearance of diverse lipoproteins from systemic circulation. The APOE gene is polymorphic, existing as three common isoforms-apoE2, apoE3, and apoE4. The APOE3 allele is the most common genotype, with 80% of the population carrying this allele. The less common E2 and E4 alleles are carried by 5%-10% and 10%-15% of the population, respectively. The difference between the ApoE isoforms is a single amino acid substitution that induces a structural change within the protein which alters the capacity to bind the low-density lipoprotein receptor and modifies lipid binding preferences. ApoE4 contains Arg at position 112 and Arg at position 158. ApoE3 contains Cys at position 112, and Arg at position 158. ApoE2 contains Cys at positions 112 and 158. In addition to the common apoE alleles, there are three rare apoE alleles (apoE1, apoE2*, and apoE2**). Compared to the other apoE proteins, apoE1 has Asp at position 127 instead of Gly and Cys at position 158 instead of Arg. ApoE2* has Cys at position 145 instead of Arg, and apoE2** has Gln at position 146 instead of Lys. ApoE as described herein encompasses all isoforms of apoE unless specified otherwise. A sequence for ApoE3 is provided in SEQ ID NO: 1:

(SEQ ID NO: 1)
MKVLWAALLVTFLAGCQAKVEQAVETEPEPELRQQTEWQSGQRWE
LALGRFWDYLRWVQTLSEQVQEELLSSQVTQELRALMDETMKELK
AYKSELEEQLTPVAEETRARLSKELQAAQARLGADMEDVCGRLVQ
YRGEVQAMLGQSTEELRVRLASHLRKLRKRLLRDADDLQKRLAVY
QAGAREGAERGLSAIRERLGPLVEQGRVRAATVGSLAGQPLQERA
QAWGERLRARMEEMGSRTRDRLDEVKEQVAEVRAKLEEQAQQIRL
QAEAFQARLKSWFEPLVEDMQRQWAGLVEKVQAAVGTSAAPVPSD
NH

ApoE structure comprises a 22 kDa N-terminal domain (NTD) (1-191) and 10 kDa C-terminal domain (CTD) (225-299) separated by the protease-sensitive hinge region. The receptor-binding domain (RBD) of apoE is located on its NTD (amino acids sequence 134-150), and forms a four-helix anti-parallel bundle. The CTD contains a lipid-binding region (LBR) with amino acids sequence of 244-272. This CTD is particularly responsible for the oligomerization of ApoE. CTD domain is also responsible for the globular shape of the ApoE via its three α-helices domains. Embodiments include the precursor protein containing an 18 amino acid leader sequence (signal peptide) and the mature protein in which this sequence is cleaved/absent.

The function-conservative variants may result from modifications and changes that may be made in the structure of the polypeptides, e.g. a polypeptide of SEQ ID NO:1, (and in the DNA sequences encoding it), and still obtain a functional molecule with desirable characteristics (e.g. ability to induce motor endplate formation, synaptogenesis, and/or induce macrophage M2 anti-inflammatory phenotypes in an injury site). In regards to induction of synaptogenesis, as demonstrated in the Examples, ApoE binds to LRP4 to induce AChR clustering. It may also bind to LRP1 to also induce clustering as ApoE has a high affinity for LRP1. In regards to induction of anti-inflammatory phenotypes, ApoE may upregulate anti-inflammatory markers. Without being limited by theory, this could be modulated via ferroptosis.

Exemplary variants are provided as SEQ ID NO: 2 (ApoE3 Christchurch variant having S at residue 154) and SEQ ID NO: 3 (ApoE3 Jacksonville variant having E at residue 236):

(SEQ ID NO: 2)
MKVEQAVETEPEPELRQQTEWQSGQRWELALGRFWDYLRWVQTLS
EQVQEELLSSQVTQELRALMDETMKELKAYKSELEEQLTPVAEET
RARLSKELQAAQARLGADMEDVCGRLVQYRGEVQAMLGQSTEELR
VSLASHLRKLRKRLLRDADDLQKRLAVYQAGAREGAERGLSAIRE
RLGPLVEQGRVRAATVGSLAGQPLQERAQAWGERLRARMEEMGSR
TRDRLDEVKEQVAEVRAKLEEQAQQIRLQAEAFQARLKSWFEPLV
EDMQRQWAGLVEKVQAAVGTSAAPVPSDNH
(SEQ ID NO: 3)
MKVEQAVETEPEPELRQQTEWQSGQRWELALGRFWDYLRWVQTLS
EQVQEELLSSQVTQELRALMDETMKELKAYKSELEEQLTPVAEET
RARLSKELQAAQARLGADMEDVCGRLVQYRGEVQAMLGQSTEELR
VRLASHLRKLRKRLLRDADDLQKRLAVYQAGAREGAERGLSAIRE
RLGPLVEQGRVRAATVGSLAGQPLQERAQAWGERLRARMEEMGSR
TRDRLDEVKEQEAEVRAKLEEQAQQIRLQAEAFQARLKSWFEPLV
EDMQRQWAGLVEKVQAAVGTSAAPVPSDNH

Accordingly, “function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared. Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wisconsin) pileup program, or any of sequence comparison algorithms such as BLAST, FASTA, etc.

The amino acid changes may be achieved by changing codons in the DNA sequence, according to Table 1.

TABLE 1

| Amino acids | | | Codons |
|---|---|---|---|
| Alanine | Ala | A | GCA, GCC, GCG, GCU |
| Cysteine | Cys | C | UGC, UGU |
| Aspartic Acid | Asp | D | GAC, GAU |
| Glutamic acid | Glu | E | GAA, GAG |
| Phenylalanine | Phe | F | UUC, UUU |
| Glycine | Gly | G | GGA, GGC, GGG, GGU |
| Histidine | His | H | CAC, CAU |
| Isoleucine | Ile | I | AUA, AUC, AUU |
| Lysine | Lys | K | AAA, AAG |
| Leucine | Leu | L | UUA, UUG, CUA, CUC, CUG, CUU |
| Methionine | Met | M | AUG |
| Asparagine | Asn | N | AAC, AAU |
| Proline | Pro | P | CCA, CCC, CCG, CCU |
| Glutamine | Gln | Q | CAA, CAG |
| Arginine | Arg | R | AGA, AGG, CGA, CGC, CGG, CGU |
| Serine | Ser | S | AGC, AGU, UCA, UCC, UCG, UCU |
| Threonine | Thr | T | ACA, ACC, ACG, ACU |
| Valine | Val | V | GUA, GUC, GUG, GUU |
| Tryptophan | Trp | W | UGG |
| Tyrosine | Tyr | Y | UAU |

For example, certain amino acids may be substituted for other amino acids in a protein structure without appreciable loss of capability to induce motor endplate formation and/or synaptogenesis. Since it is the interactive capacity and nature of a protein that defines that protein's biological functional activity, certain amino acid substitutions can be made in a protein sequence, and, of course, in its DNA encoding sequence, and nevertheless obtain a protein with like properties. It is thus contemplated that various changes may be made in the polypeptide sequences of the invention, or corresponding DNA sequences which encode said polypeptides, without appreciable loss of their biological activity. The capability for inducing motor endplate formation and/or synaptogenesis can be assessed by different techniques well-known in the art and as described herein.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

The apoE or function-conservative variant thereof as described herein is generally purified or isolated. By “purified” and “isolated” it is meant that the indicated molecule is present in the substantial absence of other biological macromolecules of the same type. The term “purified” as used herein preferably means at least 75% by weight, more preferably at least 85% by weight, still preferably at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

The polypeptides described herein may be produced by any technique known per se in the art, such as, without limitation, any chemical, biological, genetic or enzymatic technique, either alone or in combination.

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions.

Alternatively, the polypeptides can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

Polypeptides as described herein can be use in an isolated (e.g., purified) form or contained in a vector, such as a membrane or lipid vesicle (e.g. a liposome).

In some embodiments, compositions described herein contain apoE or a function-conservative variant thereof encapsulated in a decellularized extracellular matrix and/or a hydrogel. The decellularized extracellular matrix may be derived from a suitable tissue, including but not limited to, muscle, cardiac, pericardial, liver, brain, small intestine submucosa, bladder, lung, and vascular tissue. In some embodiments, a liquid form of extracellular matrix, e.g. skeletal muscle matrix, can assemble into a fibrous scaffold upon injection in vivo. The material can also be processed into a lyophilized form that only requires sterile water, PBS, or saline to resuspend prior to injection, which can provide ease of storage and use in a clinical setting. Hydrogels useful for polypeptide delivery include self-assembling peptide hydrogels, natural polymer-based hydrogels (such as alginate and chitosan), synthetic hydrogels (such as PEG), and hybrid hydrogels that combine natural and synthetic components. Polypeptides may be encapsulating in decellularized extracellular matrix or hydrogel for controlled release and to mimic the native extracellular matrix to enhance biocompatibility. The decellularized extracellular matrix or hydrogel may be stimuli-responsive materials that release polypeptides upon changes in temperature, pH, or other environmental factors.

The composition can further comprise cells, drugs, proteins, or polysaccharides. In some instances, the composition is coated on a device such as an implant. The composition can be delivered as a liquid, and in many instances, the composition can transition to a gel form after delivery. In certain embodiments, the composition is delivered as a powder or particulate.

In some embodiments, the composition can be formulated to be in liquid form at room temperature, typically 20° C. to 25° C., and in gel form at a temperature greater than room temperature or greater than 35° C. In some instances, the composition is configured to be delivered to a tissue parenterally, such as through a small gauge needle (e.g., 27 gauge or smaller). In some instances, said composition is suitable for direct implantation into a patient. The composition can be formulated either in a dry or hydrated form to be placed on or in wounds.

In some embodiments, the composition comprises native proteins or peptides. In some embodiments, the composition comprises native glycosaminoglycans. In some embodiments, the composition further comprises a population of exogenous or autologous therapeutic cells. The cells can be stem cells or other precursors of skeletal muscle cells or other cell types.

In some instances, the composition further comprises a therapeutic agent, and as such is configured as a drug delivery vehicle. In some instances, the composition is configured to coat surfaces, such as tissue culture plates or scaffolds, to culture skeletal muscle, skeletal muscle cells, or other cell types relevant to skeletal muscle repair.

A method of producing a composition is disclosed herein that comprises decellularized extracellular matrix comprising: obtaining from a subject a skeletal muscle or other suitable tissue sample having an extracellular matrix and non-extracellular matrix components; processing skeletal muscle or other tissue sample to remove the non-extracellular matrix component to obtain decellularized skeletal muscle or other tissue extracellular matrix and extracellular proteins and polysaccharides; and sterilizing the decellularized skeletal muscle or other tissue extracellular matrix. In some instances, said method is performed aseptically without sterilization. In some instances, said method further comprises the step of lyophilizing and grinding up the decellularized skeletal muscle or other tissue extracellular matrix. In some instances, said method further comprises the step of enzymatically treating, solubilizing, or suspending the decellularized skeletal muscle or other tissue extracellular matrix. In some instances, said decellularized skeletal muscle or other tissue extracellular matrix is digested with pepsin at a low pH.

In some instances, said method further comprises the step of suspending and neutralizing said decellularized skeletal muscle or other tissue extracellular matrix in a solution. In some instances, said solution is a phosphate buffered solution (PBS) or saline solution which can be injected through a high gauge needle into the desired tissue or organ. In some instances, said composition is formed into a gel at body temperature. In some instances, said composition further comprises cells, drugs, proteins or other therapeutic agents that can be delivered within or attached to the composition before, during or after gelation.

The muscle extracellular matrix can be derived from the native or natural matrix of mammalian muscle tissue. The muscle extracellular matrix can also be derived from an animal or synthetic source. In some instances, the decellularized muscle extracellular matrix is derived from native muscle tissue selected from the group consisting of human, porcine, bovine, goat, mouse, rat, rabbit, or any other mammalian or animal muscle.

The compositions comprise an extracellular matrix that is substantially decellularized. In some instances, a decellularized matrix comprises no living native cells with which the extracellular matrix naturally occurs. In some instances, a substantially decellularized matrix comprises less than 1%, 2%, 3%, 4%, 5%, 6%, 7%, 8%, 9% or 10% native cells by weight.

A composition can comprise a decellularized extracellular matrix and different tissue decellularized extracellular matrix or a synthetic or naturally occurring polymer from animal and non-animal sources (such as plants or synthetic collagens). For example, a composition herein comprises a natural polymer such as collagen, chitosan, alginate, glycosaminoglycans, fibrin, or hyaluronic acid. In another example, a composition herein comprises a synthetic polymer, for example without limitation, polyethylene glycol, poly(glycolic) acid, poly(lactic acid), poly(hydroxy acids), polydioxanone, polycaprolactone, poly(ortho esters), poly(anhydrides), polyphosphazenes, poly(amino acids), pseudo-poly(amino acids), conductive polymers (such as polyacetylene, polypyrrole, polyaniline), or polyurethane or their potential copolymers. In some instances, a composition comprises extracellular matrix and both a natural and a synthetic polymer. A composition can be a multi-material by linking an extracellular matrix and another polymer material, for example, via reaction with amines, free thiols, or short peptides that can be self-assembled with the extracellular matrix.

In some embodiments, heparin is used to covalently bond apoE to a biomaterial as described herein, e.g. a decellularized extracellular matrix, as apoE has a heparin binding site. Heparin may be immobilized to a decellularized extracellular matrix scaffold to create a binding site for sequestering apoE within the matrix. In some embodiments, an intermediate linker, such as collagen-binding peptide, is used to attach heparin to the matrix.

In some instances, a polymer of the composition is biocompatible and biodegradable and/or bioabsorbable, and can be a random copolymer, block copolymer or blend of monomers, homopolymers, copolymers, and/or heteropolymers that contain these monomers. Exemplary biodegradable or bioabsorbable polymers include, but are not limited to: polylactides, poly-glycolides, polycarprolactone, polydioxane and their random and block copolymers. A biodegradable and/or bioabsorbable polymer can contain a monomer selected from the group consisting of a glycolide, lactide, dioxanone, caprolactone, trimethylene carbonate, ethylene glycol and lysine. The biodegradable and/or bioabsorbable polymers can contain bioabsorbable and biodegradable linear aliphatic polyesters such as polyglycolide (PGA) and its random copolymer poly(glycolide-co-lactide-) (PGA-co-PLA). Other examples of suitable biocompatible polymers are polyhydroxyalkyl methacrylates including ethylmethacrylate, and hydrogels such as polyvinylpyrrolidone and polyacrylamides. Other suitable bioabsorbable materials are biopolymers which include collagen, gelatin, alginic acid, chitin, chitosan, fibrin, hyaluronic acid, dextran, polyamino acids, polylysine and copolymers of these materials. Any combination, copolymer, polymer or blend thereof of the above examples is contemplated for use according to the present invention. Such bioabsorbable materials may be prepared by known methods.

The compositions described herein can further comprise cells, drugs, proteins, or other biological material such as, but not limited to, erythropoietin (EPO), stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), stem cell factor (SCF), platelet-derived growth factor (PDGF), endothelial cell growth supplement (EGGS), colony stimulating factor (CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor matrix metalloproteinase (TIMP), interferon, interleukins, cytokines, integrin, collagen, elastin, fibrillins, fibronectin, laminin, glycosaminoglycans, hemonectin, thrombospondin, heparan sulfate, dermantan, chondroitin sulfate (CS), hyaluronic acid (HA), vitronectin, proteoglycans, transferrin, cytotactin, tenascin, and lymphokines.

The active agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The active agents described herein may be combined with pharmaceutically acceptable excipients/carriers. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The medicaments/pharmaceutical compositions may also contain preserving agents, solubilizing agents, stabilizing agents, wetting agents, emulsifiers, sweeteners, colorants, odorants, salts for the variation of osmotic pressure, buffers, coating agents or antioxidants.

In some embodiments, the composition comprises a nucleic acid encoding apoE or a function-conservative variant thereof for administration to a subject. For example, an mRNA encoding apoE or the function-conservative variant may be encapsulated in a protective delivery vehicle, e.g. lipid nanoparticles (LNPs), to shield it from degradation and facilitate entry into cells. The LNP is taken up by cells in the body, often at the injection site. Inside the cell, the mRNA is released and interacts with ribosomes to produce the polypeptide encoded by the mRNA.

Embodiments provide for the treatment of a muscle injury in a subject in need thereof, comprising administering to the subject a therapeutically effective amount of a composition comprising apolipoprotein E (apoE) or a function-conservative variant thereof. Muscle injuries may include traumatic muscle injuries (such as volumetric muscle loss) that damage both skeletal muscle fibers and intramuscular axons, where the muscle loses motor units and severs the connection to the remaining intact muscle. Other muscle injuries may arise from a number of sources, including for example: denervation; degenerative, metabolic or inflammatory neuropathy; infantile and juvenile spinal muscular atrophies; autoimmune motor neuropathy; from chronic disease, including cachexia resulting from cancer, AIDS, fasting or rhabdomyolysis; and from muscular dystrophy syndromes such as Duchenne.

In some embodiments, the muscle injury results from denervation due to nerve trauma; degenerative, metabolic or inflammatory neuropathy, e.g. Guillian-Barré syndrome; peripheral neuropathy; or nerve damage caused by environmental toxins or drugs. Muscle injury may also result from denervation due to a motor neuropathy including, for example, adult motor neuron disease, such as Amyotrophic Lateral Sclerosis (ALS or Lou Gehrig's disease); infantile and juvenile spinal muscular atrophies; and autoimmune motor neuropathy with multifocal conductor block. Muscle injury may also result from chronic disease resulting from, for example, paralysis due to stroke or spinal cord injury; skeletal immobilization due to trauma, such as, for example, fracture, ligament or tendon injury, sprain or dislocation; or prolonged bed rest. Metabolic stress or nutritional insufficiency, which may also result in muscle injury or atrophy, include the cachexia of cancer and other chronic illnesses including AIDS, fasting or rhabdomyolysis, and endocrine disorders such as disorders of the thyroid gland and diabetes. Muscle injury atrophy may also be due to a muscular dystrophy syndromes such as Duchenne, Becker, myotonic, fascioscapulohumeral, Emery-Dreifuss, oculopharyngeal, scapulohumeral, limb girdle, and congenital types, as well as the dystrophy known as Hereditary Distal Myopathy. Muscle atrophy may also be due to a congenital myopathy, such as benign congenital hypotonia, central core disease, nemalene myopathy, and myotubular (centronuclear) myopathy.

Muscle injury may also result from pressure ulcers, venous ulcers, diabetic ulcers, chronic vascular ulcers, tunneled/undermined wounds, surgical wounds (donor sites/grafts, post-Mohs surgery, post-laser surgery, podiatric, wound dehiscence), trauma wounds (abrasions, lacerations, second-degree burns and skin tears), draining wounds, inflammation, Buerger disease, atherosclerosis obliterans, and thromboangiitis obliterans, and gangrene.

The compositions described herein are useful for maintaining the integrity of a neuromuscular junction (NMJ) which refers to preserving its complex structure and crucial functions to ensure reliable communication between motor neurons and muscle fibers, enabling proper muscle contraction. This involves keeping the NMJ structurally intact, ensuring efficient and appropriate neurotransmitter release and reception, and preventing damage or degeneration from factors like toxins, disease, aging, and lack of physical activity. The compositions are particularly useful for reducing endplate fragmentation which occurs at the NMJ. Fragmentation refers to the breaking apart of the AChR clusters on the muscle fiber's surface. Nerve injury or diseases can disrupt the signals needed to maintain the structure of the NMJ endplate which leads to muscle atrophy, decreased mobility, and paralysis because the muscle can no longer receive effective signals from the nerve.

Some embodiments provide for methods of reconnecting an innervating nerve in denervated muscle tissue comprising administering a composition as described herein to maintain the integrity of the NMJ and surgically reconnecting the innervating nerve. A surgeon may use one of several known techniques for aligning the ends of the nerve and promoting regeneration, such as direct nerve repair, nerve grafting, nerve transfer, and nerve conduits.

Methods known in the art for the therapeutic delivery of agents such as proteins or nucleic acids can be used for the therapeutic delivery of a composition as described herein for treating a subject, e.g., cellular transfection, gene therapy, direct administration with a delivery vehicle or pharmaceutically acceptable carrier, indirect delivery by providing recombinant cells comprising a nucleic acid encoding a polypeptide as described herein.

Various delivery systems are known and can be used to administer the polypeptides described herein, e.g., encapsulation in liposomes, microparticles, microcapsules, recombinant cells capable of expressing the protein, receptor-mediated endocytosis, construction of a nucleic acid as part of a retroviral or other vector, etc. Methods of introduction can be enteral or parenteral and include but are not limited to intradermal, intramuscular, intraperitoneal, intravenous, subcutaneous, pulmonary, intranasal, intraocular, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local, e.g. by injection or implatantion at the site of the muscle injury, local infusion during surgery, topical application, e.g., by injection, by means of a catheter, or by means of an implant, the implant being of a porous, non-porous, or gelatinous material, including membranes, such as sialastic membranes, fibers, or commercial skin substitutes. In addition, it may be desirable to introduce the pharmaceutical compositions into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. The composition can also be delivered as a liquid, gel or in a solid formulation, such as a graft or patch or associated with a cellular scaffold or a particulate.

In another embodiment, the active agent can be delivered in a vesicle, in particular a liposome. In yet another embodiment, the active agent can be delivered in a controlled release system. In another embodiment, the active agent is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector, or by direct injection, or by use of microparticle bombardment, or coating with lipids or cell-surface receptors or transfecting agents, or by administering it in linkage to a homeobox-like peptide which is known to enter the nucleus, etc. Alternatively, a nucleic acid can be introduced intracellularly and incorporated within host cell DNA for expression, by homologous recombination.

The present disclosure encompasses the use of nucleic acids encoding the polypeptides described herein for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response.

In another aspect, the invention provides a method of treating a target site, i.e., a target cell or tissue, in a human or other animal comprising transfecting a cell with a nucleic acid encoding a tissue-specific polypeptide of the invention, wherein the nucleic acid comprises an inducible promoter operably linked to the nucleic acid encoding the polypeptide. For gene therapy procedures in the treatment or prevention of human disease, see for example, Van Brunt (1998) Biotechnology 6:1149-1154.

In some embodiments, the polypeptides described herein may be administered in combination with one or more additional compounds or therapies. The combination therapy may encompass simultaneous or alternating administration. In addition, the combination may encompass acute or chronic administration. In some embodiments, agrin is administered to the subject. In some embodiments, agrin is not administered to the subject.

A patient or subject to be treated by any of the compositions or methods of the present disclosure can mean either a human or a non-human animal, e.g. mammals, including, but not limited to dogs, horses, cats, rabbits, gerbils, hamsters, rodents, birds, aquatic mammals, cattle, pigs, camelids, and other zoological animals.

In some embodiments, the active agent is administered to the subject in a therapeutically effective amount. By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Further embodiments also provide a pharmaceutical pack or kit comprising one or more containers filled with at least one composition, polypeptide, or nucleic acid encoding a polypeptide as described herein. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE 1. APOE3 ENHANCES ACETYLCHOLINE RECEPTOR CLUSTERING VIA LRP4

The goal of the present study was to investigate pathway enrichment between myotubes and motor neurons in vitro to better understand the signaling interplay leading to NMJ formation and identify new signaling factors that may play a role in this process. RNA-sequencing results identified the involvement of apolipoprotein E (ApoE) prompting further investigation into the factor's involvement in motor endplate pre-patterning. Here we present that the isoform ApoE3, but not ApoE4, dose dependently promote synaptogenesis in vitro via the Lrp4 receptor.

### Materials & Methods

### Transwell Co-Culture

Cells were cultured as previously described (Redden et al., 2025b). C2C12 cells were plated onto a 24-well plate (#3412, Corning) in α-MEM no nucleosides full media (10% FBS with 1% P/S) at a density of 10,000 cells/cm². The following day, NSC34 motor neurons were plated in DMEM 4.5 full media on 0.4 μm pore polycarbonate transwell membranes at 10,000 cells/cm². Cells were cultured at 37° C. at 5% CO₂. Transwell inserts were moved to the C2C12 24-well plate once myoblasts reached 100% confluence and NSC34 motor neurons reached 60% confluence. Cells were the co-cultured in Neurobasal media with 4 mM L-glutamine and 1% P/S for 3 days with media changed every 24 hours. After 3 days, cell RNA was collected with TRIzol.

### Recombinant ApoE3/4 Dosage Culture

C2C12 myoblasts were plated at 10,000 cells/cm² onto 24-well plates (PCR/DNA: Corning CellBind® #3337, ICC: Ibidi® μ-plate, #82426) in α-MEM no nucleosides supplemented with 10% FBS and 1% P/S at 37C 5% CO₂. Treatment began when cells reached 100% confluence (~72 hours after plating). Recombinant human ApoE3 (APE-H5246) and ApoE4 (APE-H52H9) were acquired from ACROBiosystems (Delaware, United States). Lyophilized protein stock (100 μg) was reconstituted in 167 μL of sterile filtered ultra-pure water (stock: 600 μg/mL) for 1 hour at room temperature on the first treatment day and kept at 4° C. during the duration of the experiment. Cells received α-MEM full media supplemented with either 0 M, 10⁻⁹ M, 10⁻⁸ M, or 10⁻⁷ M recombinant ApoE3 or ApoE4 protein every 24 hours for six days then collected on the seventh day for fluorescent imaging (ICC), DNA quantification, or RT-qPCR (n=6/group, 4 groups total).

### Lrp4 Inhibitor Culture

C2C12 myoblasts were cultured as described above with treatment beginning at 100% confluence. Stock monoclonal Lrp4 antibody at 1 μg/μL (ABIN7193345) was acquired from Antibodies-online (PA, United States). Cells were provided the Lrp4 mAb at either 0 μg/well, 0.5 μg/well, 0.1 μg/well, or 0.01 μg/well every 24 hours for six days. These cells additionally received either 0 M or 10⁻⁷ M of ApoE3 supplemented into the media in addition to the anti-Lrp4 mAb Additional C2C12 groups were dose-dependently treated with ApoE3 as described in the previous section. (n=6/group, 10 groups total). AChR clusters were quantified by positive staining of α-BTX described in later sections.

### RNA-Sequencing—Library Preparation

Using TRIzol® extraction, total RNA was purified from each set of cell type lysate named C (control) or TX (co-culture treatment) generating four groups (n=6/group) of either C_C2C12, TX_C2C12, C_NSC, or TX_NSC. RNA quality was measured using a Bioanalyzer 2100 and RNA 6000 Pico® kit (Agilent Technologies, Delaware, United States). Whole RNA sequencing was performed with the MinION® sequencer (Oxford Nanopore, Oxford, United Kingdom). PCR-cDNA barcoding library preparation was performed with the SQK-PCB109 kit (Oxford Nanopore). Twelve samples per run were loaded into the Spot On® Mk1 R9.4 flow cell (Oxford Nanopore) into the MinIT®-001 device with base-calling performed by the MinKNOW® (21.05.24) and Guppy® (5.0.16) data processing toolkit.

### Bioinformatic & Pathway Enrichment Analyses

Fastq files were processed using Virginia Commonwealth University's high performance research computing core facility using minimap2 (2.21-r1071) aligning the reads to the Mus Musculus GRCm38 ENSEMBL reference genome. Using R-studio with the DESeq2 Bioconductor pipeline, gene expression was compared from treatment against control for each respective cell group (C2C12_C vs C2C12_TX, NSC_C vs NSC_TX). Genes were considered differentially expressed if the absolute log 2 fold-change ≥1-fold and had a p<0.05. Pathway enrichment comparisons were performed between Gene Ontology Consortium (geneontology.org/), Reactome (reactome.org/), and Molecular Signature Hallmark (gsea-msigdb.org/gsea/index.jsp) databases. Additional graphs and pathway plots were produced using RNASeqChef (Etoh and Nakao, 2023) (imeg-ku.shinyapps.io/RNAseqChef/) using cutoffs of FC=1 and false discovery ratio (FDR)=0.05 computed with DESeq2 and the IHW procedure.

### Immunocytochemistry (ICC)

For fixation and permeabilization, cells were treated with ice cold methanol for 5-minutes at −20° C. then washed three times with 1×PBS. A stain solution containing Alexa Fluor® 647 Bungarotoxin (1:250 from 1 mg/mL stock, Invitrogen, CA, United States), phalloidin 488 conjugate (1:50, Cayman Chemical, MI, United States), and DAPI 33258 (1:20,000 from 10 mg/mL stock, Invitrogen). Samples were incubated in stain solution for 1 hour at room temperature on a rocker protected from light then washed with 1×PBS three times. Images were captured using the Zeiss® 980 LSM using the parameters shown in Table 2. Counts, lengths, and widths were measured in ImageJ® (Schindelin et al., 2012) and orientation distributions with the OrientationJ® plugin v2.0.5 (Rezakhaniha et al., 2012).

TABLE 2
Parameters for image capture

| Parameter | Value |
|---|---|
| Microscope | Zeiss ® 980 |
| Image Resolution | 1024 × 1024 px |
| Bits per Pixel | BitsPerPixel = 16; PixelType = uint16 |
| Pixel Dwell Time | 0.768 μs |
| Exposure Time | 150.0 ms (approx.) |
| Magnification | 20x |
| Scan Speed | 6 u |
| Averaging | AveragingNumber = 8; AveragingMode = Line |
| Channels | Muliti-Track: AF647, AF488, DAPI |
| Emission (nm) | AF647: 668 |
| | AF488: 517 |
| | DAPI: 465 |
| Excitation (nm) | AF647: 653 |
| | AF488: 493 |
| | DAPI: 353 |

### DNA Quantification Assay

A solution of 0.05% Triton® X-100 solution was used to lyse collected cells. Following cell scraping in lysate, the lysate was then transferred to 1.5 mL tubes. Samples were then sonicated on ice (40 amps, 5 seconds). The Quant-iT® PicoGreen® dsDNA assay kit (#P7589, Thermo Fisher Scientific) was performed following manufacturer guidelines. Results were measured using a spectrophotometer at 504 excitation and 531 emission (BioTek Take3, BioTek Synergy H1, VT, United States).

### RT-qPCR

Using 100 ng of RNA per sample, cDNA was generated using the high-capacity cDNA RT kit (#4368814, Thermo Fisher Scientific). RT-qPCR was performed with Power Sybr® Green using a QuantStudio® 3 (Thermo Fisher Scientific).

### Statistical Analysis

GraphPad Prism® 10.0 (GraphPad, CA, United States) was used for any statistical tests (not including sequencing analysis) with data shown as mean±SEM. Unpaired t-tests were used in assays with two groups. Test results with p-values <0.05 were considered significant and Prism® displays significance as *<0.05, **<0.01, ***<0.001. A one-way ANOVA with a Tukey post-hoc test was used to compare multiple groups with columns containing different letters representing statistical significance (p<0.05).

### Results

C2C12 Myotubes Differentially Expressed 171 Genes when Co-Cultured with NSC-34 Motor Neurons

C2C12 myotubes were co-cultured for three days with NSC-34 motor neurons in a transwell co-culture system. RNA was collected and isolated separately after 3 days in co-culture. Total RNA sequencing was performed using the MinION® sequencer. Acquired reads were aligned and analyzed using a combination of R and Matlab®. Heatmap analysis of DE genes showed minor variance in the downregulated genes and similar expression between samples in the upregulated genes (data not shown). Of the 15,225 genes read in sequencing, 60 were upregulated while 111 were downregulated using a fold-change cut-off of 1 and a p-value cut-off of 0.05. Volcano plotting provided a preliminary evaluation of differentially regulated genes with notable genes such as Myod1 matching previous RT-qPCR results. Applying a false discovery ratio cut-off of 0.05, 24 genes were identified as key differential regulators in the co-cultured myotubes.

### Motor Neuron Signaling Promotes Myogenic and Glycolytic Pathways in Myotubes

Multiple pathway libraries were referenced all showing co-culture enriched pathways associated with myogenesis and muscle contraction further confirming the pro-myogenic results identified in our previous experiments. The bi-plot component identified Mb (Myoglobin) and Sppl (Osteopontin) as the top two genes weighting the PCA. Additionally, the weighted biplot shows that the upregulation of genes was the primary differentiator between groups (data not shown). Additionally, the Principal component analysis (PCA) of group clustering and corroborative Scree plot confirm the majority of variance is explainable in the first component and groups are distinctly clustered. Pathway analysis for NSC-34 motor neurons showed an upregulation in glycolytic and ACh-related genes (data not shown).

### ErbB Pathway Downregulation Reveal an Associated ApoE Exocytosis Feedback Loop Via ADAM17 Cleavage Enzyme

Reactome pathway analysis showed an upregulation of neurotransmitter pathways such as acetylcholine mediated pathways, whereas ErbB-mediated signaling was downregulated. These findings prompted a search into these downregulated pathways using the Reactome analysis tools, showing that NRG-ErbB pathways were less active in co-culture. Investigation of the pathway revealed a known Apoe-Erbb: S80 feedback loop. According to the pathway analysis, intracellular cleavage of ErbB by ADAM proteases would downregulate the receptor's associated signals, and implied a connection between ErbB cleavage and increased ApoE secretion.

ApoE and Non-Canonical Wnt Signaling Associated with Spontaneous Aneural AChR Clustering is Upregulated in Co-Cultured Myotubes

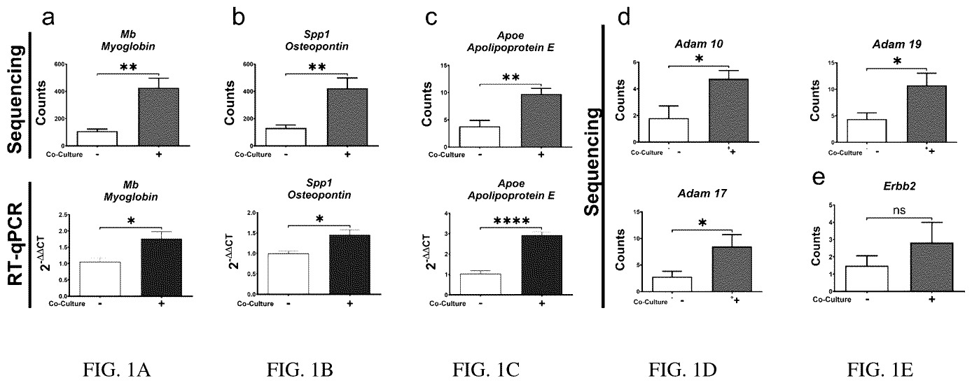

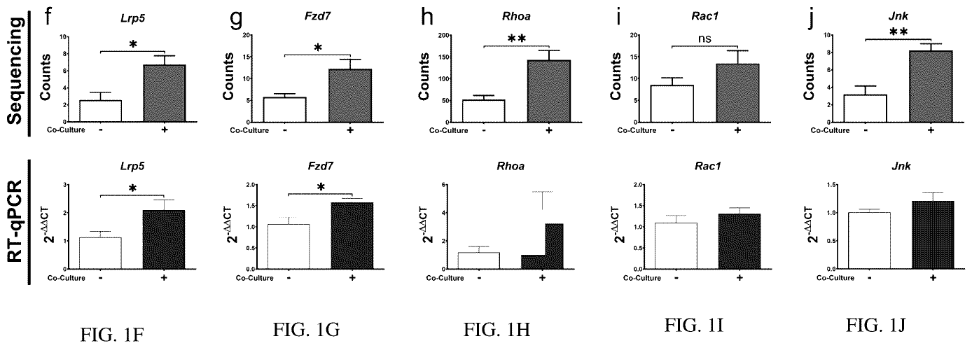

We first used RT-qPCR to confirm Sppl and Mb normalized counts measured during sequencing (FIG. 1A,B). Next, we determined that Apoe expression was upregulated in our sequencing data and this increase was validated with RT-qPCR (FIG. 1C). We then examined Adam related genes from our Reactome pathway analysis and determined that Adam10/17/19 were upregulated in our sequencing data (FIG. 1D). Adam 12 and 15 were unchanged (data not shown). To complete this pathway analysis, we measured Erbb2 counts from our sequencing data and found that while average counts were elevated in co-cultures, we did not observe a significant increase (FIG. 1E). Wnt associated receptor genes Lrp5 (FIG. 1F) and Fzd7 (FIG. 1G) were upregulated in sequencing, and validated using RT-qPCR. Downstream Rhoa (FIG. 1H) was also upregulated whereas Racl (FIG. 1I) was unaffected. Jnk (c-jun N terminal kinase gene promoter), a downstream factor of Wnt, was upregulated (FIG. 1J), indicating a non-canonical Wnt-Rhoa activation with potential PKC activity. However, RT-qPCR analysis did not confirm the increases observed in sequencing for Rhoa and Jnk.

We investigated Wnt receptor gene expression levels further using our sequencing data. While Lrp5 increased in response to motor neuron co-culture, Lrp4 and Lrp6 co-culture levels were similar to controls (data not shown). It should be noted that the average counts in control and treatment groups were much higher for Lrp4 (100-175) compared to Lrp5 (2-7). While Fzd2 was unchanged, Fzd7 increased in co-culture groups (data not shown). Next, we compared non-canonical and canonical Wnt signaling mRNA and determined that non-canonical signaling increased in response to co-culture (data not shown).

### Recombinant ApoE3 Dose Dependently Increases Chrng Expression

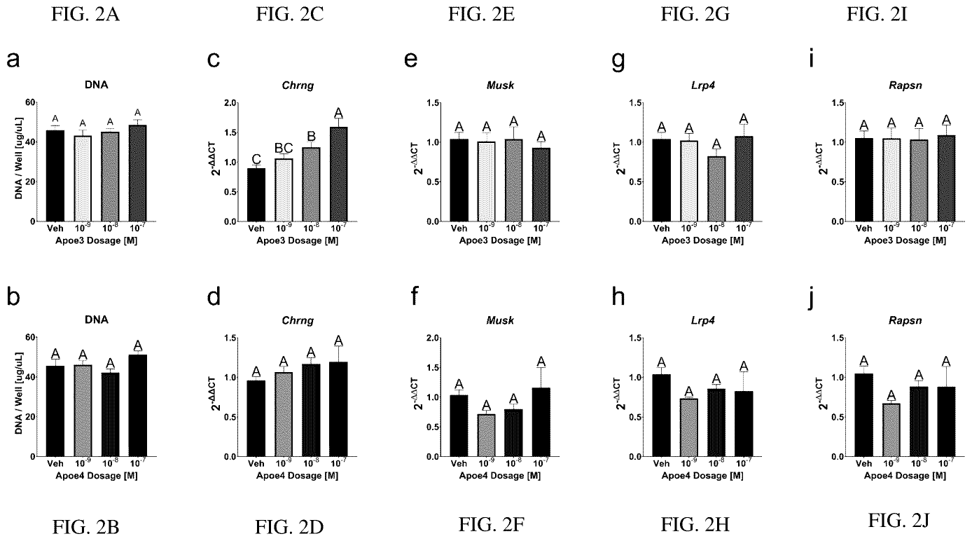

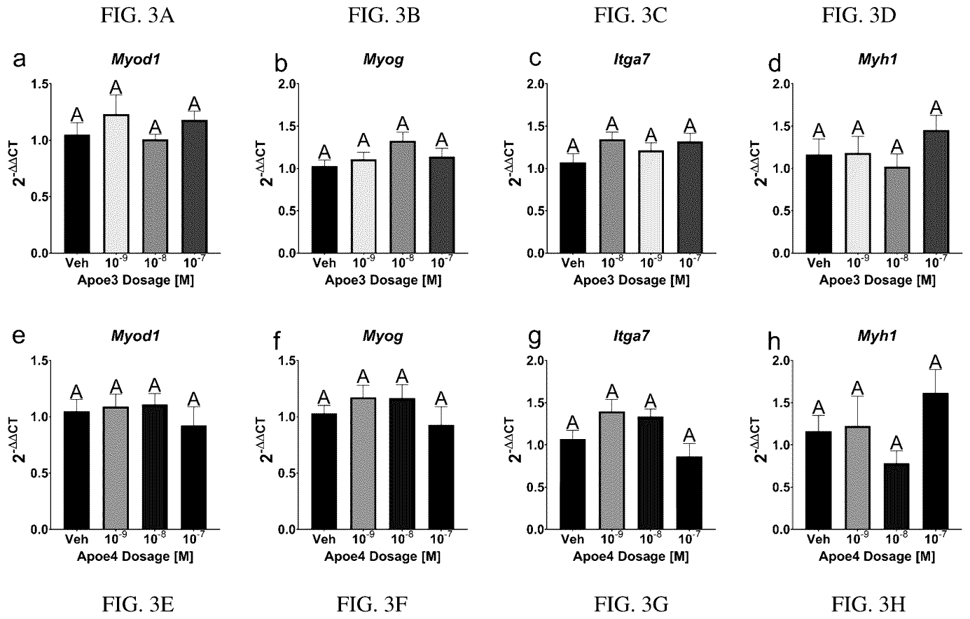

Sequencing data and reactome pathway analysis prompted our investigation into the role of ApoE. C2C12 myoblasts received ApoE3 or ApoE4 doses at 10⁻⁹, 10⁻⁸, 10⁻⁷ M every day for six days throughout myogenesis. DNA levels were unaffected by ApoE3 or ApoE4, suggesting the treatment did not impact proliferation (FIGS. 2A,B). Chrng, encoding the immature AChR-γ subunit, was dose dependently increased by recombinant ApoE3 (FIG. 2C), while ApoE4 had no effect (FIG. 2D). Lrp4, Musk, and Rapsn expression, associated with AChR clustering and stabilization, were unaffected (FIGS. 2E-J) by ApoE3 or ApoE4 treatment. When we changed ApoE dosing to a later time point, we did not observe the same increase in Chrng (data not shown). Additionally, neither isoform affected myogenic markers suggesting a specific effect on AChR clustering (FIG. 3).

### ApoE3 Induces Aneural AChR Clustering Via Lrp4

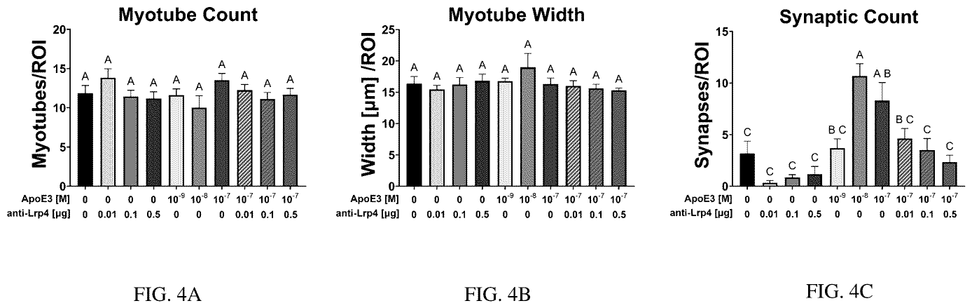

We theorized the effect of ApoE3 acted via the Lrp4 receptor due to the increased mRNA counts observed in FIG. 1. To test this theory, we blocked the N-terminal and first propeller domain with an anti-Lrp4 mAb (AA22-189) responsible for binding ApoE. C2C12 cells received either fresh media, anti-Lrp4 mAb (0.5, 0.1, or 0.01 μg), ApoE3 (10⁻⁷, 10⁻⁸, 10⁻⁹), or ApoE3 with anti-Lrp4 mAb every day for 6 days. AChR clustering dose dependently increased in groups receiving ApoE3 (data not shown). Myotubes treated with 0 M anti-Lrp4 showed no observable changes in AChR clustering. In contrast, groups treated with 10⁻⁷ M ApoE3 and anti-Lrp4 mAb exhibited decreased AChR clustering. As the anti-Lrp4 dosage decreased, AChR clustering increased (data not shown). No changes in overall myofiber size were observed in any group, where myotube counts and widths were unaffected by either treatment (FIGS. 4A,B) consistent with the RT-qPCR data measuring myogenic markers (FIG. 3). Synaptic counting confirmed our observations in FIG. 2, where 10⁻⁸ and 10⁻⁷ M ApoE3 doses showed the highest number of AChR clusters compared to controls and anti-Lrp4 mAb treated groups (FIG. 4C).

### Discussion

Here we report that ApoE3 binds to Lrp4 to dose-dependently increase AChR clustering on C2C12 myotubes independent of agrin. Our study was prompted due to previous observations from multiple in vivo volumetric muscle injury studies that highlight Apoe as a differentially expressed target (Redden et al., 2025a). These in vivo experiments corroborated our in vitro muscle-nerve cross-talk co-culture results that also demonstrated elevated Apoe mRNA and protein. Prior to investigating ApoE directly, total RNA-seq and pathway analysis was performed to more deeply investigate the impact of motor neuron signaling on myotube expression. These data indicated differential regulation of non-canonical Wnt and ErbB pathways which led to the discovery of a known Erb-ApoE feedback loop, providing a causal link between synaptogenesis and ApoE and supporting a basis for additional research. After confirming increased Apoe expression, we treated C2C12 myotubes with recombinant ApoE. ApoE is found in muscle as both ApoE3 and ApoE4. Thus, we treated myotubes with ApoE3 and ApoE4 in order to determine their role in muscle regeneration and synaptogenesis. Of the two isoforms, only ApoE3 was observed to increase Chrng and synaptogenesis despite the isoforms sharing similar homology. Finally, we showed ApoE3 induced AChR clustering acted via Lrp4 by competitively blocking the N-terminal and 1st propeller binding domain on the receptor with an anti-Lrp4 mAb, allowing us to observe dose dependent changes to AChR clustering.

Of note, the NSC34 neurons in co-culture with C2C12 showed responsive changes in their expression. NSC34 in co-culture displayed increases in Mif, a gene associated with central nervous system neuro-inflammation modulation (Nasiri et al., 2020), but also plays important roles in peripheral nerve regeneration (Nishio et al., 2002). Additionally, genes implicated in mitochondrial regulation (Higd1a) (Hayashi et al., 2015), (Pkm) (Liang et al., 2017), (Gapdh) (Lee et al., 2016)) and axogenesis (Rpl36) (Fusco et al., 2021) were upregulated in NSC34s co-cultured with myotubes. However, in this study, we focused on the effects of co-culture on the C2C12 myotubes.

Comparative pathway enrichment between multiple datasets (e.g. Reactome, GO biological, KEGG, and MSigDB Hallmark) showed analogous upregulation of myogenic and muscle contraction pathways. ACh mediated signaling, ECM mediated signaling, signal transduction, and glycolytic associated pathways were significantly upregulated in motor neuron co-cultured myotubes, but ErbB associated pathways decreased. This is in contrast to our previous understanding of ErbB (Schmidt et al., 2011) signaling in synaptogenesis since NRG-ErbB signaling assists in AChR stabilization and turnover which is further validated by animal knockout models dying in embryo (Falls, 2003; Meyer and Birchmeier, 1995; Sandrock et al., 1997). These contrasting findings prompted a search into ErbB pathways leading to the eventual discovery that knockdown of ErbB signal transduction can lead to ErbB: s80 fragments in the nucleoplasm capable of generating a feedback loop in expression increasing secretion of ApoE (Wali et al., 2014), but these findings have never been demonstrated in skeletal muscle. This is especially important in NRG-1/ErbB signaling where metalloproteases can cleave mRNA splice variants of ErbB (Elenius et al., 1997) allowing the intracellular S80 fragment to translocate to the nucleus further regulating transcription (Ni et al., 2001). Previous literature has also shown ADAM19 protease may modulate NMJ formation via ErbB signaling in response to electrical stimulation (Fukazawa et al., 2013) as well as ADAM19/ErbB driven ephrin-A5-Eph signaling (Yumoto et al., 2008). ADAM17 and ADAM10 have also been shown to modulate EGFR family/ErbB signaling in multiple tissues (Dombernowsky et al., 2015; Luo et al., 2011; Zbodakova et al., 2021).

Notably, in this study we show Adam9/10/17/19 are increased in motor neuron co-cultured myotubes, but due complex enzymatic actions of ADAM proteins, it is not possible to distinguish which is responsible for ErbB cleaving using the current sequencing data. However, upregulated expression of multiple Adams and a downregulation of ErbB pathways due to co-culture with motor neurons suggests an interaction during synaptogenesis. Future studies are needed to identify the pathway responsible for Adam upregulation, protein synthesis, and which, ADAM proteases are cleaving ErbB into S80. It is possible that Apoe transcription is regulated by ErbB cleavage via ADAM proteases. If correct, this pathway may help regulate aneural AChR clustering via secretion of ApoE3 for autocrine signaling to Lrp4 in the absence of neural agrin. Additionally, it may help explain why aneural clusters are dispersed along the myotube prior to neural invasion rather than centralized on the fiber. However, a full determination of ErbB and Adam pathways will require additional experiments to detect protein interactions, track S80 fragment nuclear-localization, and measure transcriptional regulation.

To date, it is well-understood that agrin is secreted by the motor neuron and supports post-synaptic development of the NMJ, but this process requires the presence of the motor neuron. Additionally, agrin knockout models in mice were still able to form AChR clusters before innervation, indicating that agrin was not required in aneural pre-patterning (Lin et al., 2001). While our untreated and anti-Lrp4 treated myotube cultures spontaneously developed AChR clusters, those clusters were significantly lower than ApoE3 treatments. These low AChR levels may involve alternative synaptic pathways such as Wnt-related synaptogenesis (Koles and Budnik, 2012). Non-canonical Wnts (Jing et al., 2021) act in concert with adenomatous polyposis coli (APC) and agrin to increase AChR clustering, and is believed to contribute to cytoskeletal organization critical for NMJ formation and stabilization (Reilein and Nelson, 2005; Temburni et al., 2004; Wang et al., 2003). Additionally, β-catenin downstream of Wnt signaling has been shown to bind to rapsyn and alter AChR clustering both in vivo (Li et al., 2008) and in vitro (Zhang et al., 2007). The spontaneous AChR clustering via Wnt findings are consistent with our sequencing data showing increases Rhoa and Jnk expression associated with non-canonical Wnt signaling in co-cultured myotubes. To test Wnt inhibition in combination with anti-Lrp4 mAb treatments, we briefly considered using RKI-1447, but non-canonical Wnt inhibition has been shown to diminish Lrp4 expression (Jing et al., 2021) and directly impede myogenesis due to its effect on Rho-ROCK pathways (Nishiyama et al., 2004), and would produce inconclusive results. In addition, while Lrp5, a receptor in Wnt signaling, was elevated in co-cultures, the number of mRNA counts was relatively low. In contrast, Lrp4 counts were higher, suggesting a stronger role in AChR clustering. For these reasons, we chose to limit the scope to Lrp4-ApoE interactions.

Lrp4 is a part of the lipoprotein receptor super family (LDLrs) (Li et al., 2001), and plays critical roles in bone remodeling (Collette et al., 2012), NMJ homeostasis (Weatherbee et al., 2006), and central nervous system plasticity (Mosca et al., 2017). The receptor has several binding sites allowing it to interact with multiple proteins at the same time, but at the NMJ it is best known for its dynamic interaction between agrin and MuSK. When this 1:1:1 complex forms, the complex creates a 2:2:2 heterodimer with a secondary complex allowing for phosphorylation activating downstream acetylcholine receptor clustering (Sun et al., 2023). Lrp4 expression is essential for healthy NMJ development in embryo shown by knockout mice present with transmission defects similar to congenital myasthenic syndrome (CMS) (Ohkawara et al., 2014) and myasthenia gravis (MG) (Rivner et al., 2020).

To effectively block the Lrp4 receptor's binding with ApoE3, antibody dosages were estimated based on anti-Lrp4 antibody serum titers from patients with Myasthenia Gravis (Tzartos et al., 2014) and dilutions dosing C2C12 cells with 1:100 mouse derived sera containing IgG for Lrp4 (Shen et al., 2013). Additionally, due to Lrp4 receptor's multiple propeller domains capable of binding ApoE (Herz and Strickland, 2001) and agrin (Kim et al., 2008; Zhang et al., 2008), an antibody was selected that targets both the N-terminal and the first propeller binding region (AA 21-189) responsible for initial ligand binding (Zhang et al., 2011; Zong et al., 2012). This was done with the intent of competitively blocking ligand binding without directly inhibiting MuSK-Lrp4 dimerization in the propeller domains closer to the transmembrane. Additionally, an IgG1 ab was selected since patients with anti-Lrp4 MG exhibit expression of this subclass of antibody at higher rates (Liu et al., 2011).

ApoE3 and E4 isoforms only differ in their N-terminal with E4 containing arginine whereas E3 contains cystine (Dong and Weisgraber, 1996; Sivashanmugam and Wang, 2009). While the N-terminal is the primary LDL receptor binding site, the C-terminal, which is conserved between isoforms, may play a role in lipidating ApoE allowing the N-terminal to bind to receptors (Chen et al., 2011; Weisgraber, 1994). Interestingly, lipidation is also an important step in myomaker orientation helping facilitate fusogenic protein interactions (Millay et al., 2016). These lipidation mechanisms may in part explain why we observed that a single dose of ApoE at late stage myogenesis has a null effect on synaptogenesis suggesting ApoE driven synaptogenesis can occur during myogenesis but not maturation.

ApoE3 dosages used in the present study are similar to agrin doses used in previous literature (~Oct. 10, 2010-8 M) (Marangi et al., 2001; Rimer, 2010; Weston et al., 2000). While the exact physiological concentrations around the NMJ are difficult to determine, both ApoE and agrin co-localize to the NMJ at high densities compared to surrounding muscle tissue (Akaaboune et al., 1994; Godfrey et al., 1984). The comparative binding affinity of agrin and ApoE3 to Lrp4 is unknown but may play synergistic roles in synapse formation and stability. Competitive binding immuno-assays would be required to determine which signaling factor preferentially binds to Lrp4 and cyro-EM assessing the conformational changes to the Lrp4-MuSK-ApoE3 complex (assuming MuSK associates with Lrp4 and ApoE3) would provide insight into their interaction; this has been performed already with agrin published in 2023 (Sun et al., 2023). Lastly, muscle agrin has been shown to be inefficient at inducing AChR clusters (Kim et al., 2008; Zhang et al., 2008). Thus, production and secretion of ApoE3 in muscle highlights its use as an early driver of aneural synaptogenesis alongside laminins and ColQ in the absence of neural agrin.

Amyloid precursor protein (APP), a transmembrane protein transcriptionally regulated by ApoE, can also bind to Lrp4 and enhance agrin induced AChR clustering (Choi et al., 2013). While still requiring agrin, these data, alongside our present research, highlights the dynamic role and binding availability of Lrp4 opening considerations into other proteoglyco- and lipo-proteins potentially playing a role in synaptogenesis. Other proteoglycans such as perlecan (Kamimura et al., 2013; Noonan et al., 1991) have been shown to interact with Lrp4 at the NMJ highlighting the complexity of Lrp4 interactions and an importance for future research to open its scope outside of agrin induced AChR clustering. Our present study highlights the role of ApoE3 as an aneural AChR clustering signaling factor.

### Conclusion

In summary, we show ApoE stimulated synaptogenesis is isoform dependent and recombinant ApoE3 delivery is capable of dose dependently inducing AChR clusters. Pathway analysis suggests ApoE secretion may be regulated by feedback loop driven by nuclear localization of ErbB: S80 fragments increasing transcription of ApoE. We assert that ApoE3 is a regulator in NMJ formation and possibly acts as autocrine signal to induce early aneural AChR clusters prior to innervation and neural agrin.

### EXAMPLE 2

Methods: 6 week old C57BL/6 (WT), B6.129P2-Apoe^{tml/Une}/J (Apoe KO), and B6.Cg-Apoe^{em2(APOE*3)Adiuj}/J (Apoe3 KI) male mice were used to test muscle force. Muscle force was measured using an Aurora Scientific 1300A Whole Animal System for Mice. Mice were anesthetized using 2-4% isoflurane/400 mL/min O₂. Twitch and tetany muscle forces were measured by placing the paw firmly onto the footpad and stabilizing the knee. Two electrodes were inserted in the subdermal space of the gastrocnemius with the positive electrode proximal to the negative electrode. Stimulating voltages, muscle stretch, and signal frequency were optimized for each limb prior to recording data. Twitch stimulations were performed first with <1 ms electrical pulses and a ~1s rest period between pulses. Tetany measurements were performed with 500 -ms stimulations and a 120-s resting period. Maximum force was defined as the peak force generated within the data set. N=6 animals per group tested.

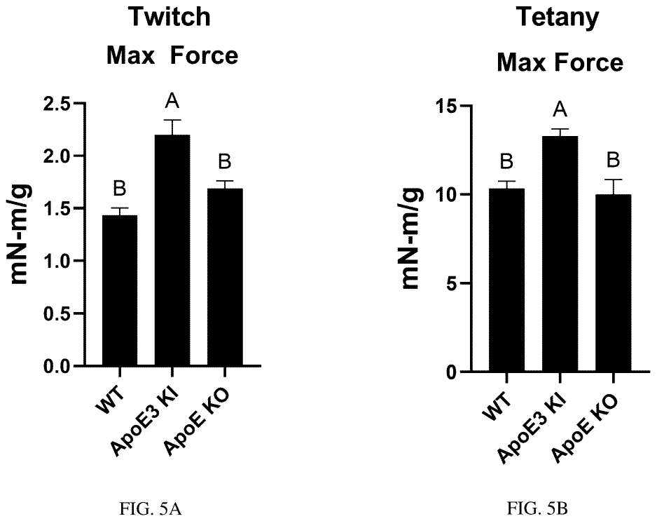

Results: Data from muscle force tests were collected and analyzed using the Dynamic Muscle Control and Analysis Software. Peak twitch and peak tetanic force was significantly higher in Apoe3 KI animals compared to WT and KO animals, while WT and KO were similar to each other for both twitch and tetany (FIG. 5A-B). These results suggest that ApoE3 caused a functional change in muscle force generation, and indicates that ApoE3 treatment could enhance muscle force in newly regenerated fibers.

### EXAMPLE 3

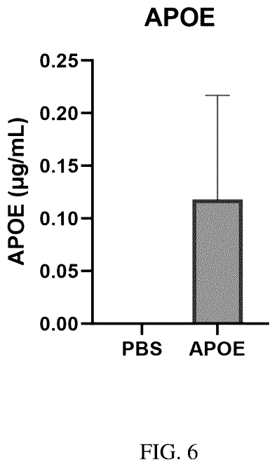

Human recombinant ApoE was adsorbed onto the surface of a decellularized muscle matrix (DMM) scaffold to test its ability to bind without the use of crosslinking reagents. DMM was incubated with 35.3 μg/mL ApoE3 at 37° C. for 24 hours. Next, DMM-ApoE was washed in 1×PBS and incubated for 7 more days at 37° C. in PBS to remove any ApoE that was unbound. ApoE was then measured using an ApoE ELISA. Human recombinant ApoE was elevated in DMM incubated with ApoE (FIG. 6).

### EXAMPLE 4

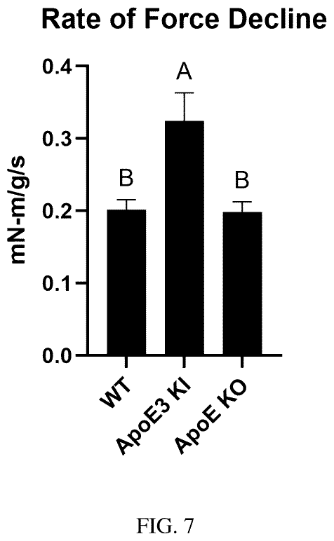

ApoE3 changed muscle fatiguability. WT, ApoE3 KI, and ApoE KO mice were subjected to muscle fatigue testing using the same setup as described in Example 2. Muscles were stimulated using the optimized tetanic force parameters 300 times with 1s rest between simulations. ApoE3 KI mice exhibited greater fatigue compared to WT and ApoE KO (FIG. 7).

### REFERENCES

Akaaboune, M., M. Villanova, B. W. Festoff, M. Verdière-Sahuqué, and D. Hantaï. 1994. Apolipoprotein E expression at neuromuscular junctions in mouse, rat and human skeletal muscle. FEBS Lett. 351:246-248.
Anderson, M. J., and M. W. Cohen. 1977. Nerve-induced and spontaneous redistribution of acetylcholine receptors on cultured muscle cells. J Physiol. 268:757-773.
Arnold, A. S., M. Christe, and C. Handschin. 2012. A functional motor unit in the culture dish: co-culture of spinal cord explants and muscle cells. J Vis Exp.
Barik, A., B. Zhang, G. S. Sohal, W. C. Xiong, and L. Mei. 2014. Crosstalk between Agrin and Wnt signaling pathways in development of vertebrate neuromuscular junction. Dev Neurobiol. 74:828-838.
Bowen, D. C., J. Sugiyama, M. Ferns, and Z. W. Hall. 1996. Neural agrin activates a high-affinity receptor in C2 muscle cells that is unresponsive to muscle agrin. J Neurosci. 16:3791-3797.
Brown, M. C., J. K. Jansen, and D. Van Essen. 1976. Polyneuronal innervation of skeletal muscle in new-born rats and its elimination during maturation. J Physiol. 261:387-422.
Bubuioc, A. M., A. Kudebayeva, S. Turuspekova, V. Lisnic, and M. A. Leone. 2021. The epidemiology of myasthenia gravis. J Med Life. 14:7-16.
Chen, J., Q. Li, and J. Wang. 2011. Topology of human apolipoprotein E3 uniquely regulates its diverse biological functions. Proc Natl Acad Sci USA. 108:14813-14818.
Choi, H. Y., Y. Liu, C. Tennert, Y. Sugiura, A. Karakatsani, S. Kröger, E. B. Johnson, R. E. Hammer, W. Lin, and J. Herz. 2013. APP interacts with LRP4 and agrin to coordinate the development of the neuromuscular junction in mice. Elife. 2:e00220.
Collette, N. M., D. C. Genetos, A. N. Economides, L. Xie, M. Shahnazari, W. Yao, N. E. Lane, R. M. Harland, and G. G. Loots. 2012. Targeted deletion of Sost distal enhancer increases bone formation and bone mass. Proc Natl Acad Sci USA. 109:14092-14097.
Dahlin, L. B., and M. Wiberg. 2017. Nerve injuries of the upper extremity and hand. EFORT Open Rev. 2:158-170.
Dahm, L. M., and L. T. Landmesser. 1988. The regulation of intramuscular nerve branching during normal development and following activity blockade. Dev Biol. 130:621-644.
Dombernowsky, S. L., J. Samsøe-Petersen, C. H. Petersen, R. Instrell, A. M. Hedegaard, L. Thomas, K. M. Atkins, S. Auclair, R. Albrechtsen, K. J. Mygind, C. Fröhlich, M. Howell, P. Parker, G. Thomas, and M. Kveiborg. 2015. The sorting protein PACS-2 promotes ErbB signalling by regulating recycling of the metalloproteinase ADAM17. Nat Commun. 6:7518.
Dong, L. M., and K. H. Weisgraber. 1996. Human apolipoprotein E4 domain interaction. Arginine 61 and glutamic acid 255 interact to direct the preference for very low density lipoproteins. J Biol Chem. 271:19053-19057.
Elenius, K., G. Corfas, S. Paul, C. J. Choi, C. Rio, G. D. Plowman, and M. Klagsbrun. 1997. A novel juxtamembrane domain isoform of HER4/ErbB4. Isoform-specific tissue distribution and differential processing in response to phorbol ester. J Biol Chem. 272:26761-26768.
Engel, A. G., X. M. Shen, D. Selcen, and S. M. Sine. 2015. Congenital myasthenic syndromes: pathogenesis, diagnosis, and treatment. Lancet Neurol. 14:420-434.
Etoh, K., and M. Nakao. 2023. A web-based integrative transcriptome analysis, RNAseqChef, uncovers the cell/tissue type-dependent action of sulforaphane. J Biol Chem. 299:104810.
Falls, D. L. 2003. Neuregulins and the neuromuscular system: 10 years of answers and questions. J Neurocytol. 32:619-647.
Fox, M. A., J. R. Sanes, D. B. Borza, V. P. Eswarakumar, R. Fässler, B. G. Hudson, S. W. John, Y. Ninomiya, V. Pedchenko, S. L. Pfaff, M. N. Rheault, Y. Sado, Y. Segal, M. J. Werle, and H. Umemori. 2007. Distinct target-derived signals organize formation, maturation, and maintenance of motor nerve terminals. Cell. 129:179-193.
Fukazawa, T., M. Matsumoto, T. Imura, E. Khalesi, T. Kajiume, Y. Kawahara, K. Tanimoto, and L. Yuge. 2013. Electrical stimulation accelerates neuromuscular junction formation through ADAM19/neuregulin/ErbB signaling in vitro. Neurosci Lett. 545:29-34.
Fusco, C. M., K. Desch, A. R. Dörrbaum, M. Wang, A. Staab, I. C. W. Chan, E. Vail, V. Villeri, J. D. Langer, and E. M. Schuman. 2021. Neuronal ribosomes exhibit dynamic and context-dependent exchange of ribosomal proteins. Nat Commun. 12:6127.
Gemza, A., C. Barresi, J. Proemer, J. Hatami, M. Lazaridis, and R. Herbst. 2022. Internalization of Muscle-Specific Kinase Is Increased by Agrin and Independent of Kinase-Activity, Lrp4 and Dynamin. Front Mol Neurosci. 15:780659.
Godfrey, E. W., R. M. Nitkin, B. G. Wallace, L. L. Rubin, and U. J. McMahan. 1984. Components of Torpedo electric organ and muscle that cause aggregation of acetylcholine receptors on cultured muscle cells. J Cell Biol. 99:615-627.

Hayashi, T., Y. Asano, Y. Shintani, H. Aoyama, H. Kioka, O. Tsukamoto, M. Hikita, K. Shinzawa-Itoh, K. Takafuji, S. Higo, H. Kato, S. Yamazaki, K. Matsuoka, A. Nakano, H. Asanuma, M. Asakura, T. Minamino, Y. Goto, T. Ogura, M. Kitakaze, I. Komuro, Y. Sakata, T. Tsukihara, S. Yoshikawa, and S. Takashima. 2015. Higdla is a positive regulator of cytochrome c oxidase. Proc Natl Acad Sci USA. 112:1553-1558.
Herz, J., and D. K. Strickland. 2001. LRP: a multifunctional scavenger and signaling receptor. J Clin Invest. 108:779-784.
Hicks, C. W., and E. Selvin. 2019. Epidemiology of Peripheral Neuropathy and Lower Extremity Disease in Diabetes. Curr Diab Rep. 19:86.
Hubbard, S. R., and K. Gnanasambandan. 2013. Structure and activation of MuSK, a receptor tyrosine kinase central to neuromuscular junction formation. Biochim Biophys Acta. 1834:2166-2169.
Jing, H., P. Chen, T. Hui, Z. Yu, J. Zhou, E. Fei, S. Wang, D. Ren, X. Lai, and B. Li. 2021. Synapse-specific Lrp4 mRNA enrichment requires Lrp4/MuSK signaling, muscle activity and Wnt non-canonical pathway. Cell Biosci. 11:105.
Kamimura, K., K. Ueno, J. Nakagawa, R. Hamada, M. Saitoe, and N. Maeda. 2013. Perlecan regulates bidirectional Wnt signaling at the Drosophila neuromuscular junction. J Cell Biol. 200:219-233.
Keller-Peck, C. R., M. K. Walsh, W. B. Gan, G. Feng, J. R. Sanes, and J. W. Lichtman. 2001. Asynchronous synapse elimination in neonatal motor units: studies using GFP transgenic mice. Neuron. 31:381-394.
Kim, N., A. L. Stiegler, T. O. Cameron, P. T. Hallock, A. M. Gomez, J. H. Huang, S. R. Hubbard, M. L. Dustin, and S. J. Burden. 2008. Lrp4 is a receptor for Agrin and forms a complex with MuSK. Cell. 135:334-342.
Koles, K., and V. Budnik. 2012. Wnt signaling in neuromuscular junction development. Cold Spring Harb Perspect Biol. 4.
Lee, F. H., P. Su, Y. F. Xie, K. E. Wang, Q. Wan, and F. Liu. 2016. Disrupting GluA2-GAPDH Interaction Affects Axon and Dendrite Development. Sci Rep. 6:30458.
Li, X. M., X. P. Dong, S. W. Luo, B. Zhang, D. H. Lee, A. K. Ting, H. Neiswender, C. H. Kim, E. Carpenter-Hyland, T. M. Gao, W. C. Xiong, and L. Mei. 2008. Retrograde regulation of motoneuron differentiation by muscle beta-catenin. Nat Neurosci. 11:262-268.
Li, Y., J. Cam, and G. Bu. 2001. Low-density lipoprotein receptor family: endocytosis and signal transduction. Mol Neurobiol. 23:53-67.
Liang, J., R. Cao, X. Wang, Y. Zhang, P. Wang, H. Gao, C. Li, F. Yang, R. Zeng, P. Wei, D. Li, W. Li, and W. Yang. 2017. Mitochondrial PKM2 regulates oxidative stress-induced apoptosis by stabilizing Bcl2. Cell Res. 27:329-351.
Lin, W., R. W. Burgess, B. Dominguez, S. L. Pfaff, J. R. Sanes, and K. F. Lee. 2001. Distinct roles of nerve and muscle in postsynaptic differentiation of the neuromuscular synapse. Nature. 410:1057-1064.
Lin, W., B. Dominguez, J. Yang, P. Aryal, E. P. Brandon, F. H. Gage, and K. F. Lee. 2005. Neurotransmitter acetylcholine negatively regulates neuromuscular synapse formation by a Cdk5-dependent mechanism. Neuron. 46:569-579.
Liu, Y., Y. Luo, L. Shen, R. Guo, Z. Zhan, N. Yuan, R. Sha, W. Qian, Z. Wang, Z. Xie, W. Wu, and Y. Feng. 2020. Splicing Factor SRSF1 Is Essential for Satellite Cell Proliferation and Postnatal Maturation of Neuromuscular Junctions in Mice. Stem Cell Reports. 15:941-954.
Liu, Y., W. Wang, and J. Li. 2011. Evaluation of serum IgG subclass concentrations in myasthenia gravis patients. Int J Neurosci. 121:570-574.
Luo, X., M. Prior, W. He, X. Hu, X. Tang, W. Shen, S. Yadav, S. Kiryu-Seo, R. Miller, B. D. Trapp, and R. Yan. 2011. Cleavage of neuregulin-1 by BACE1 or ADAM10 protein produces differential effects on myelination. J Biol Chem. 286:23967-23974.
Marangi, P. A., J. R. Forsayeth, P. Mittaud, S. Erb-Vögtli, D. J. Blake, M. Moransard, A. Sander, and C. Fuhrer. 2001. Acetylcholine receptors are required for agrin-induced clustering of postsynaptic proteins. EMBO J. 20:7060-7073.
Meyer, D., and C. Birchmeier. 1995. Multiple essential functions of neuregulin in development. Nature. 378:386-390.
Millay, D. P., D. G. Gamage, M. E. Quinn, Y. L. Min, Y. Mitani, R. Bassel-Duby, and E. N. Olson. 2016. Structure-function analysis of myomaker domains required for myoblast fusion. Proc Natl Acad Sci USA. 113:2116-2121.
Mosca, T. J., D. J. Luginbuhl, I. E. Wang, and L. Luo. 2017. Presynaptic LRP4 promotes synapse number and function of excitatory CNS neurons. Elife. 6.
Nasiri, E., R. Sankowski, H. Dietrich, A. Oikonomidi, P. T. Huerta, J. Popp, Y. Al-Abed, and M. Bacher. 2020. Key role of MIF-related neuroinflammation in neurodegeneration and cognitive impairment in Alzheimer's disease. Mol Med. 26:34.
Ni, C. Y., M. P. Murphy, T. E. Golde, and G. Carpenter. 2001. gamma-Secretase cleavage and nuclear localization of ErbB-4 receptor tyrosine kinase. Science. 294:2179-2181.
Nishimune, H., G. Valdez, G. Jarad, C. L. Moulson, U. Müller, J. H. Miner, and J. R. Sanes. 2008. Laminins promote postsynaptic maturation by an autocrine mechanism at the neuromuscular junction. J Cell Biol. 182:1201-1215.
Nishio, Y., J. Nishihira, T. Ishibashi, H. Kato, and A. Minami. 2002. Role of macrophage migration inhibitory factor (MIF) in peripheral nerve regeneration: anti-MIF antibody induces delay of nerve regeneration and the apoptosis of Schwann cells. Mol Med. 8:509-520.
Nishiyama, T., I. Kii, and A. Kudo. 2004. Inactivation of Rho/ROCK signaling is crucial for the nuclear accumulation of FKHR and myoblast fusion. J Biol Chem. 279:47311-47319.
Nitkin, R. M., M. A. Smith, C. Magill, J. R. Fallon, Y. M. Yao, B. G. Wallace, and U. J. McMahan. 1987. Identification of agrin, a synaptic organizing protein from Torpedo electric organ. J Cell Biol. 105:2471-2478.
Noble, J., C. A. Munro, V. S. Prasad, and R. Midha. 1998. Analysis of upper and lower extremity peripheral nerve injuries in a population of patients with multiple injuries. J Trauma. 45:116-122.
Noonan, D. M., A. Fulle, P. Valente, S. Cai, E. Horigan, M. Sasaki, Y. Yamada, and J. R. Hassell. 1991. The complete sequence of perlecan, a basement membrane heparan sulfate proteoglycan, reveals extensive similarity with laminin A chain, low density lipoprotein-receptor, and the neural cell adhesion molecule. J Biol Chem. 266:22939-22947.
Ohkawara, B., M. Cabrera-Serrano, T. Nakata, M. Milone, N. Asai, K. Ito, M. Ito, A. Masuda, Y. Ito, A. G. Engel, and K. Ohno. 2014. LRP4 third β-propeller domain mutations cause novel congenital myasthenia by compromising agrin-mediated MuSK signaling in a position-specific manner. Hum Mol Genet. 23:1856-1868.
Pęziński, M., P. Daszczuk, B. S. Pradhan, H. Lochmüller, and T. J. Prószyński. 2020. An improved method for culturing myotubes on laminins for the robust clustering of postsynaptic machinery. Sci Rep. 10:4524.
Redden, J. T., D. J. Cohen, L. C. Olson, G. Bendale, J. E. Isaacs, Z. Schwartz, and M. J. McClure. 2025a. Neurotization of decellularized muscle graft increases de novo type I slow muscle fiber formation and large fiber size frequency. Acta Biomater. 191:244-259.
Redden, J. T., S. Kothe, D. J. Cohen, Z. Schwartz, and M. J. McClure. 2025b. Trophic Factors in Muscle-Nerve Cross-Talk Signaling Augment Muscle Fiber and Motor Endplate Development. J Cell Physiol. 240:e70013.
Redfern, P. A. 1970. Neuromuscular transmission in new-born rats. J Physiol. 209:701-709.
Reilein, A., and W. J. Nelson. 2005. APC is a component of an organizing template for cortical microtubule networks. Nat Cell Biol. 7:463-473.
Rezakhaniha, R., A. Agianniotis, J. T. Schrauwen, A. Griffa, D. Sage, C. V. Bouten, F. N. van de Vosse, M. Unser, and N. Stergiopulos. 2012. Experimental investigation of collagen waviness and orientation in the arterial adventitia using confocal laser scanning microscopy. Biomech Model Mechanobiol. 11:461-473.
Rimer, M. 2010. Modulation of agrin-induced acetylcholine receptor clustering by extracellular signal-regulated kinases 1 and 2 in cultured myotubes. J Biol Chem. 285:32370-32377.
Rivner, M. H., B. M. Quarles, J. X. Pan, Z. Yu, J. F. Howard, A. Corse, M. M. Dimachkie, C. Jackson, T. Vu, G. Small, R. P. Lisak, J. Belsh, I. Lee, R. J. Nowak, V. Baute, S. Scelsa, J. A. Fernandes, Z. Simmons, A. Swenson, R. Barohn, R. B. Sanka, C. Gooch, E. Ubogu, J. Caress, M. Pasnoor, H. Xu, and L. Mei. 2020. Clinical features of LRP4/agrin-antibody-positive myasthenia gravis: A multicenter study. Muscle Nerve. 62:333-343.
Sandrock, A. W., S. E. Dryer, K. M. Rosen, S. N. Gozani, R. Kramer, L. E. Theill, and G. D. Fischbach. 1997. Maintenance of acetylcholine receptor number by neuregulins at the neuromuscular junction in vivo. Science. 276:599-603.
Sanes, J. R., and J. W. Lichtman. 1999. Development of the vertebrate neuromuscular junction. Annu Rev Neurosci. 22:389-442.
Sanes, J. R., and J. W. Lichtman. 2001. Induction, assembly, maturation and maintenance of a postsynaptic apparatus. Nat Rev Neurosci. 2:791-805.
Schindelin, J., I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak, and A. Cardona. 2012. Fiji: an open-source platform for biological-image analysis. Nat Methods. 9:676-682.
Schmidt, N., M. Akaaboune, N. Gajendran, I. Martinez-Pena y Valenzuela, S. Wakefield, R. Thurnheer, and H. R. Brenner. 2011. Neuregulin/ErbB regulate neuromuscular junction development by phosphorylation of α-dystrobrevin. J Cell Biol. 195:1171-1184.
Shen, C., Y. Lu, B. Zhang, D. Figueiredo, J. Bean, J. Jung, H. Wu, A. Barik, D. M. Yin, W. C. Xiong, and L. Mei. 2013. Antibodies against low-density lipoprotein receptor-related protein 4 induce myasthenia gravis. J Clin Invest. 123:5190-5202.
Shimizu, K., H. Kassai, Y. Kamei, K. Yamamoto, T. Nagashima, T. Maekawa, H. Akiyama, and H. Honda. 2022. Alignment of Skeletal Muscle Cells Facilitates Acetylcholine Receptor Clustering and Neuromuscular Junction Formation with Co-Cultured Human iPSC-Derived Motor Neurons. Cells. 11.
Sivashanmugam, A., and J. Wang. 2009. A unified scheme for initiation and conformational adaptation of human apolipoprotein E N-terminal domain upon lipoprotein binding and for receptor binding activity. J Biol Chem. 284:14657-14666.
Song, Y., and R. Balice-Gordon. 2008. New dogs in the dogma: Lrp4 and Tid1 in neuromuscular synapse formation. Neuron. 60:526-528.
Southam, K. A., A. E. King, C. A. Blizzard, G. H. McCormack, and T. C. Dickson. 2013. Microfluidic primary culture model of the lower motor neuron-neuromuscular junction circuit. J Neurosci Methods. 218:164-169.
Stoklund Dittlau, K., E. N. Krasnow, L. Fumagalli, T. Vandoorne, P. Baatsen, A. Kerstens, G. Giacomazzi, B. Pavie, E. Rossaert, J. Beckers, M. Sampaolesi, P. Van Damme, and L. Van Den Bosch. 2021. Generation of Human Motor Units with Functional Neuromuscular Junctions in Microfluidic Devices. J Vis Exp.
Sun, H., H. Shang, X. Pan, and M. Li. 2023. Structural insights into the assembly and energy transfer of the Lhcb9-dependent photosystem I from moss Physcomitrium patens. Nat Plants. 9:1347-1358.
Temburni, M. K., M. M. Rosenberg, N. Pathak, R. McConnell, and M. H. Jacob. 2004. Neuronal nicotinic synapse assembly requires the adenomatous polyposis coli tumor suppressor protein. J Neurosci. 24:6776-6784.
Tourovskaia, A., T. F. Kosar, and A. Folch. 2006. Local induction of acetylcholine receptor clustering in myotube cultures using microfluidic application of agrin. Biophys J. 90:2192-2198.
Tzartos, J. S., P. Zisimopoulou, M. Rentzos, N. Karandreas, V. Zouvelou, P. Evangelakou, A. Tsonis, T. Thomaidis, G. Lauria, F. Andreetta, R. Mantegazza, and S. J. Tzartos. 2014. LRP4 antibodies in serum and CSF from amyotrophic lateral sclerosis patients. Ann Clin Transl Neurol. 1:80-87.
Umbach, J. A., K. L. Adams, C. B. Gundersen, and B. G. Novitch. 2012. Functional neuromuscular junctions formed by embryonic stem cell-derived motor neurons. PLOS One. 7:e36049.
Vilmont, V., B. Cadot, G. Quanounou, and E. R. Gomes. 2016. A system for studying mechanisms of neuromuscular junction development and maintenance. Development. 143:2464-2477.
Wali, V. B., J. W. Haskins, M. Gilmore-Hebert, J. T. Platt, Z. Liu, and D. F. Stern. 2014. Convergent and divergent cellular responses by ErbB4 isoforms in mammary epithelial cells. Mol Cancer Res. 12:1140-1155.
Wang, J., Z. Jing, L. Zhang, G. Zhou, J. Braun, Y. Yao, and Z. Z. Wang. 2003. Regulation of acetylcholine receptor clustering by the tumor suppressor APC. Nat Neurosci. 6:1017-1018.
Weatherbee, S. D., K. V. Anderson, and L. A. Niswander. 2006. LDL-receptor-related protein 4 is crucial for formation of the neuromuscular junction. Development. 133:4993-5000.
Weisgraber, K. H. 1994. Apolipoprotein E: structure-function relationships. Adv Protein Chem. 45:249-302.
Weston, C., B. Yee, E. Hod, and J. Prives. 2000. Agrin-induced acetylcholine receptor clustering is mediated by the small guanosine triphosphatases Rac and Cdc42. J Cell Biol. 150:205-212.
Xie, T., G. Xu, Y. Liu, B. Quade, W. Lin, and X. C. Bai. 2023. Structural insights into the assembly of the agrin/LRP4/MuSK signaling complex. Proc Natl Acad Sci USA. 120:e2300453120.
Yumoto, N., S. Wakatsuki, T. Kurisaki, Y. Hara, N. Osumi, J. Frisén, and A. Sehara-Fujisawa. 2008. Meltrin beta/ADAM19 interacting with EphA4 in developing neural cells participates in formation of the neuromuscular junction. PLoS One. 3:e3322.
Zbodakova, O., K. Chalupsky, L. Sarnova, P. Kasparek, M. Jirouskova, M. Gregor, and R. Sedlacek. 2021. ADAM10 and ADAM17 regulate EGFR, c-Met and TNF RI signalling in liver regeneration and fibrosis. Sci Rep. 11:11414.
Zhang, B., S. Luo, X. P. Dong, X. Zhang, C. Liu, Z. Luo, W. C. Xiong, and L. Mei. 2007. Beta-catenin regulates acetylcholine receptor clustering in muscle cells through interaction with rapsyn. J Neurosci. 27:3968-3973.
Zhang, B., S. Luo, Q. Wang, T. Suzuki, W. C. Xiong, and L. Mei. 2008. LRP4 serves as a coreceptor of agrin. Neuron. 60:285-297.
Zhang, W., A. S. Coldefy, S. R. Hubbard, and S. J. Burden. 2011. Agrin binds to the N-terminal region of Lrp4 protein and stimulates association between Lrp4 and the first immunoglobulin-like domain in muscle-specific kinase (MuSK). J Biol Chem. 286:40624-40630.
Zong, Y., B. Zhang, S. Gu, K. Lee, J. Zhou, G. Yao, D. Figueiredo, K. Perry, L. Mei, and R. Jin. 2012. Structural basis of agrin-LRP4-MuSK signaling. Genes Dev. 26:247-258.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of treating a muscle injury in a subject in need thereof, comprising administering to the subject a therapeutically effective amount of a composition comprising apolipoprotein E (apoE) or a function-conservative variant thereof.

2. The method of claim 1, wherein the muscle injury comprises denervated muscle tissue.

3. The method of claim 2, further comprising reconnecting an innervating nerve in the denervated muscle tissue after administration of the composition.

4. The method of claim 1, wherein the composition comprises apoE or a function-conservative variant thereof encapsulated in a decellularized extracellular matrix.

5. The method of claim 4, wherein the decellularized extracellular matrix is a decellularized muscle matrix.

6. The method of claim 1, wherein the composition comprises a hydrogel containing apoE or a function-conservative variant thereof.

7. The method of claim 1, wherein the composition comprises a nucleic acid encoding apoE or a function-conservative variant thereof.

8. The method of claim 1, wherein the composition is injected or implanted at a site of the muscle injury.

9. The method of claim 1, wherein the apoE is an apolipoprotein E2 (apoE2), apolipoprotein E3 (apoE3), or apolipoprotein E4 (apoE4) isoform.

10. The method of claim 1, wherein the function-conservative variant has a sequence at least 90% identical to SEQ ID NO: 1.


SEQUENCE LISTING (3 sequences)

SEQ ID NO: 1 - protein, 317 aa, Homo sapiens [sequence omitted]

SEQ ID NO: 2 - protein, 300 aa, Homo sapiens
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 3 - protein, 300 aa, Homo sapiens
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAEVRAKLEEQAQQIRLQAEAFQARLKSWFEPLVEDMQRQWAGLVEKVQAAVGTSAAPVPSDNH